(19) United States
(12) Patent Application Publication
(10) Pub. No.: US 2023/0233173 A1
(43) Pub. Date: Jul. 27, 2023

(54) PLEURAL EFFUSION PRE-SCREENING SYSTEM

(71) Applicant: HILL-ROM SERVICES PTE. LTD., Singapore (SG)

(72) Inventors: Gee Han PNG, Singapore (SG); Yaolong LOU, Singapore (SG); Aaron A. AYU, Singapore (SG); Kok Boon R. ONG, Singapore (SG); Karen MULLERY, St. Paul, MN (US); Susan E. MALARET, Chanhassen, MN (US); Stacey REULAND, St. Paul, MN (US); Eng Keong TAY, Singapore (SG); Chau Chong YE, Singapore (SG); Justin LIEW, Singapore (SG); Baoyi WU, Singapore (SG)

(21) Appl. No.: 18/158,733

(22) Filed: Jan. 24, 2023

Related U.S. Application Data
(60) Provisional application No. 63/303,621, filed on Jan. 27, 2022.

(51) Int. Cl. A61B 7/00; A61B 7/04; A61B 5/00

(52) U.S. Cl. CPC A61B 7/003; A61B 7/04; A61B 5/7246; A61B 5/6831; A61B 2562/0204

(57) ABSTRACT

A pleural effusion pre-screening system may be used to administer a percussive treatment to a patient's chest and/or back, sense the respiratory sounds from the percussive treatment, and analyze those respiratory sounds. The pleural effusion pre-screening system may have a high frequency chest wall oscillation (HFCWO) vest which includes at least one adjustable strap.

### CROSS-REFERENCE TO RELATED APPLICATIONS

This application claims priority under 35 U.S.C. § 119(e) to U.S. Provisional Patent Application No. 63/303,621, filed Jan. 27, 2022, which is expressly incorporated by reference herein.

### BACKGROUND

The present disclosure relates to a percussion therapy apparatus that is operable to provide high frequency chest wall oscillation and also provide pleural effusion pre-screening system. More specifically, the present disclosure relates to a pleural effusion pre-screening system that can be worn by a patient to predict the presence of pleural effusion in the patient's lungs.

Patients who may have pleural effusion undergo pre-screening by a medical care professional, such as a doctor, where the doctor will give the patient a physical exam by tapping on the patient's chest and listening for pleural effusion. In order to confirm the existence of pleural effusion, the doctor then prescribes imaging tests. This adds repetitive steps to the patient's treatment, and prescribing imaging tests without more certainty can be costly for the medical professional and/or patient.

Some patients who wear high frequency chest wall oscillation (HFCWO) vests may experience physical discomforts due to the weight, sizing, and positioning of the vest. This discourages the patient from wearing the HFCWO vest for necessary treatment. The most direct way to access lung health condition is to visualize the lung by imaging. Lung health is usually provided by chest images from x-ray, computed tomography (CT), and magnetic resonance imaging (MRI) techniques. These techniques are suitable for visualizing the airways and lung pathology. However, the cumbersome and unwieldy equipment required to prepare these images require that the images be captured at the equipment and must not be impeded by foreign objects such as clothing, jewelry, or the like. Electrical impedance tomography (EIT) is an imaging technology that can be implemented to provide portability to patients, but still requires the removal of clothing and the like to apply electrodes on the patient's skin on their chest and back. Vibration response imaging (VRI) by acoustic signals is another technique that is portable to the patient, but also suffers the drawback of attaching multiple sensors to the patient's skin.

The use of HFCWO techniques are known to provide ongoing pulmonary therapy that may be varied in intensity, frequency, and location to provide therapy tailored to a particular patient. For example, the Monarch® Airway Clearance System available from Hill-Rom, Inc., Batesville, Ind., provides mobility with targeted kinetic energy and airflow to thin and mobilize secretions from the airways. The use of such a therapy can be optimized by using images of the lungs/airways to target the provision of therapy to those areas that are in most need of therapy. However, the use of the therapy must be interrupted to allow for images of the lungs/airways to be gathered to provide information for targeting the therapy.

Thus, there exists a need for an assessment tool that provides outcome measures that allows for frequent monitoring, allows for an equipment-to-patient approach, provides regional/localized information on lung function, and eliminates the subjective nature of assessment.

### SUMMARY

The present disclosure includes one or more of the features recited in the appended claims and/or the following features which, alone or in any combination, may comprise patentable subject matter.

According to a first aspect of the present disclosure, a pleural effusion pre-screening system comprises a garment, a first percussion device, a first sensor, and a controller. The garment is wearable by a person. The first percussion device is secured to the garment and positioned in a predetermined location relative to the body of the person. The first sensor is secured to the garment and positioned in the predetermined location relative to the body of the person. The controller includes a processor and a memory device including instructions that, when executed by the processor, activates the first sensor to begin recording sound data from the patient's respiratory system, activates the first percussion device to simulate manual chest tapping, collect sound data from the patient's respiratory system, and sends the sound data to a post processing feature.

In some embodiments of the first aspect, the system deactivates the first percussion device and the sensor is deactivated before sending the sound data to the post processing feature.

In some embodiments of the first aspect, the sensor is a sound transducer. In other embodiments, the sensor is a microphone. In still other embodiments, the sensor is a digital stethoscope.

In some embodiments of the first aspect, the system further comprises a plurality of percussion devices and a plurality of sensors associated with each of the percussion devices.

In some embodiments of the first aspect, the memory device includes further instructions that, when executed by the processor, activates a second sensor to begin recording sound data from the patient's respiratory system, activates a second percussion device to simulate manual chest tapping, collect sound data from the patient's respiratory system, and sends the sound data from the second sensor to a post processing feature.

In some embodiments of the first aspect, the system deactivates the second percussion device and the second sensor is deactivated before sending the sound data to the post processing feature.

In some embodiments of the first aspect, the memory device includes further instructions that, when executed by the processor, sequentially activates each additional sensor to begin recording sound data from the patient's respiratory system, activates an additional percussion device associated with the respective additional sensor to simulate manual chest tapping, collects sound data from the patient's respiratory system, deactivates the additional sensor and the additional percussion device, and sends the sound data from the additional sensor to a post processing feature.

In some embodiments of the first aspect, the memory device includes further instructions that, when executed by the processor, processes the sound data to extract sound features, classifies the sound features, compares the classified sound features to threshold settings, and, based on the comparison of the sound features to the threshold settings, predicts the likelihood that the person is experiencing pleural effusion.

In some embodiments of the first aspect, the memory device includes further instructions that, when executed by the processor, processes the sound data to extract sound features, classifies the sound features, compares the classified sound features to threshold settings, and, based on the comparison of the sound features to the threshold settings, predicts the likelihood that the person is experiencing pleural effusion.

In some embodiments of the first aspect, the memory device includes further instructions that, when executed by the processor, applies a digital signal filter to the sound data prior to performing the extraction of sound features.

According to a second aspect of the present disclosure, a method of performing pleural effusion pre-screening comprises the steps of: (i) positioning a garment having a plurality of percussion devices and a plurality of sensors on a person so that each percussion device and a respective sensor is positioned on a target region of the person; (ii) activating a sensor to begin recording sound data from the patient's respiratory system; (iii) activating a respective percussion device associated with the respective sensor to simulate manual chest tapping; (iv) collecting sound data from the person respiratory system; (v) storing the sound data; (vi) repeating (ii) through (v) for each sensor and respective percussion device; (vii) forwarding all of the sound data to a post processing operation.

In some embodiments of the second aspect, the method further comprises applying a digital signal filter to the sound data.

In some embodiments of the second aspect, the method further comprises extracting sound features from the sound data.

In some embodiments of the second aspect, the method further comprises classifying the sound features.

In some embodiments of the second aspect, the method further comprises comparing the classified sound features to threshold settings.

In some embodiments of the second aspect, the method further comprises predicting the likelihood of pleural effusion in the person.

According to a third aspect of the present disclosure, a high frequency chest wall oscillation (HFCWO) system comprises an adjustable garment, at least one first percussion device positioned on a first side of the garment, and at least one second percussion device positioned on a second side of the garment. The garment is adjustable between a first position locating the first and second percussion devices to target an upper zone of the person's lungs and a second position locating the first and second percussion device to target a lower zone of the person's lungs.

In some embodiments of the third aspect, the first and second percussion devices are operable to vary the power delivered to the person's lungs such that a first power can be delivered at each respective zone.

In some embodiments of the third aspect, the adjustable garment includes an adjustable strap.

In some embodiments of the third aspect, the adjustable strap comprises at least one adjustable shoulder strap and at least one adjustable underarm strap.

In some embodiments of the third aspect, the HFCWO system further comprises a sensor associated with each respective percussion device. The HFCWO system may also further comprise a controller including a processor and a memory device. The memory device may include instructions that, when executed by the processor, sequentially and independently activates each sensor to begin recording sound data from the patient's respiratory system, activates the percussion device associated with the respective sensor to simulate manual chest tapping, collects sound data from the patient's respiratory system, deactivates the additional sensor and the additional percussion device, repeats the sequential and independent activation of the sensor, respective percussion device, data collection and deactivation of the sensor and respective percussion device for each set of sensors and percussion devices, and sends the sound data from the additional sensor to a post processing feature.

In some embodiments of the third aspect, the memory device includes further instructions that, when executed by the processor, processes the sound data to extract sound features, classifies the sound features, compares the classified sound features to threshold settings, and, based on the comparison of the sound features to the threshold settings, predicts the likelihood that the person is experiencing pleural effusion.

In some embodiments of the third aspect, the memory device includes further instructions that, when executed by the processor, applies a digital signal filter to the sound data prior to performing the extraction of sound features.

In some embodiments of the third aspect, the percussion devices administer a therapy force of no more than 18 Newton at a frequency no more than 20 Hertz.

In some embodiments of the third aspect, the therapy force is administered at a range of 3 to 18 Newton and the frequency of the therapy force is at a range of 5 to 20 Hertz.

According to a fourth aspect of the present disclosure, a method of providing high frequency chest wall oscillation therapy comprises the step of positioning a garment with percussion devices on a person so that the percussion devices are positioned with a first percussion device positioned over the person's chest at a respective upper zone of the patient's lungs and a second percussion device positioned over the person's back at the respective upper zone of the patient's lungs. The method also comprises the step of delivering therapy to the patient at a first setting for each percussion device. The method also comprises the step of re-positioning the garment so that the first percussion device is positioned over the person's chest at a respective lower zone of the patient's lungs and the second percussion device is positioned over the person's back at the respective lower zone of the patient's lungs. The method also comprises the step of delivering therapy to the patient at a second setting for each percussion device.

In some embodiments of the fourth aspect, the first setting for each percussion device is equal to the second setting for each percussion device.

In some embodiments of the fourth aspect, the first setting for each percussion device is not equal to the second setting for each percussion device.

In some embodiments of the fourth aspect, repositioning the garment includes adjusting an adjustable strap of the garment.

The method of the fourth aspect may further comprise the steps of (i) positioning a plurality of sensors on the patient so that each percussion device has a respective sensor positioned on a target region of the person; (ii) activating a sensor to begin recording sound data from the patient's respiratory system; (iii) activating a respective percussion device associated with the respective sensor to simulate manual chest tapping; (iv) collecting sound data from the person respiratory system; (v) storing the sound data; (vi) repeating (ii) through (v) for each sensor and respective percussion device; and/or (vii) forwarding all of the sound data to a post processing operation.

In some embodiments of the fourth aspect, steps (i) through (vi) are performed in each of the first and second locations.

The method of the fourth aspect may further comprise the step of applying a digital signal filter to the sound data.

The method of the fourth aspect may further comprise the steps of extracting sound features from the sound data.

The method of the fourth aspect may further comprise classifying the sound features.

The method of the fourth aspect may further comprise comparing the classified sound features to threshold settings.

The method of the fourth aspect may further comprise predicting the likelihood of pleural effusion in the person.

According to the disclosed embodiments, a pleural effusion pre-screening system may be used to administer a percussive treatment to a patient's chest and/or back, sense the respiratory sounds from the percussive treatment, and analyze those respiratory sounds. The pleural effusion pre-screening system may have a high frequency chest wall oscillation (HFCWO) vest which includes at least one adjustable strap.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1A is a front view of a high frequency chest wall oscillation (HFCWO) system of the present disclosure worn by a patient;

FIG. 1B is a back view of the HFCWO system of FIG. 1A;

FIG. 2 is a view similar to FIG. 1A, showing the positioning of a pleural effusion pre-screening system superimposed over a patient's respiratory system;

FIG. 3 is a front view of target vibration zones over a patient's chest;

FIG. 4A is a front view of another embodiment of the HFCWO system in a first position worn by a patient;

FIG. 4B is a back view of the HFCWO system of FIG. 4A;

FIG. 5A is a front view of the HFCWO system of FIGS. 4A and 4B in a second position worn by a patient;

FIG. 5B is a back view of the HFCWO system of FIGS. 4A and 4B in a second position worn by a patient;

FIG. 6A is a front perspective view of a voice coil actuator;

FIG. 6B is a cross-section of the same view of FIG. 6A of the voice coil actuator;

FIG. 7 is a block diagram illustrating the control system of a pleural effusion pre-screening systems of the present disclosure; and

FIG. 8 is a flow chart illustrating instructions of a memory device in a controller of a pleural-effusion pre-screening system.

### DETAILED DESCRIPTION

Generally, healthy persons can expectorate their normal build-up of mucous, phlegm, and/or the like within their respiratory systems. Sufferers of excessive respiratory build-up and/or reduce expectoration capacity can require assistance in properly freeing such build-up from respiratory systems. Physically freeing, dislodging, and/or loosening the build-up can assist in proper expectoration.

Percussive therapy can effectively assist proper expectoration in an efficient and comfortable manner. Percussive therapy includes repeated percussive force to the patient to physically assist dislodging of the build-up. Manual percussive force should be performed only by a trained practitioner and can be physically demanding to the practitioner. Moreover, percussive force can be tiring and/or uncomfortable to the patient. Efficient and precise administration of percussive force can improve the patient's comfort and endurance in receiving percussion therapy and can improve the effectiveness of percussion therapy to dislodge build-up.

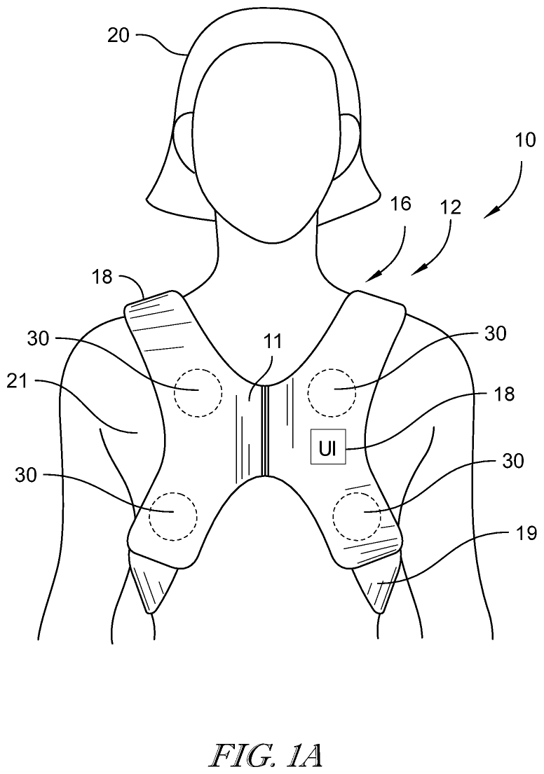

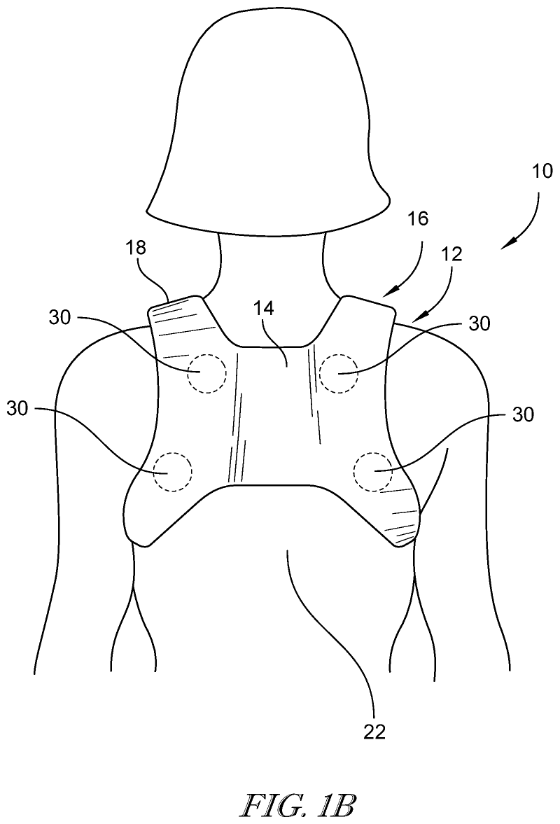

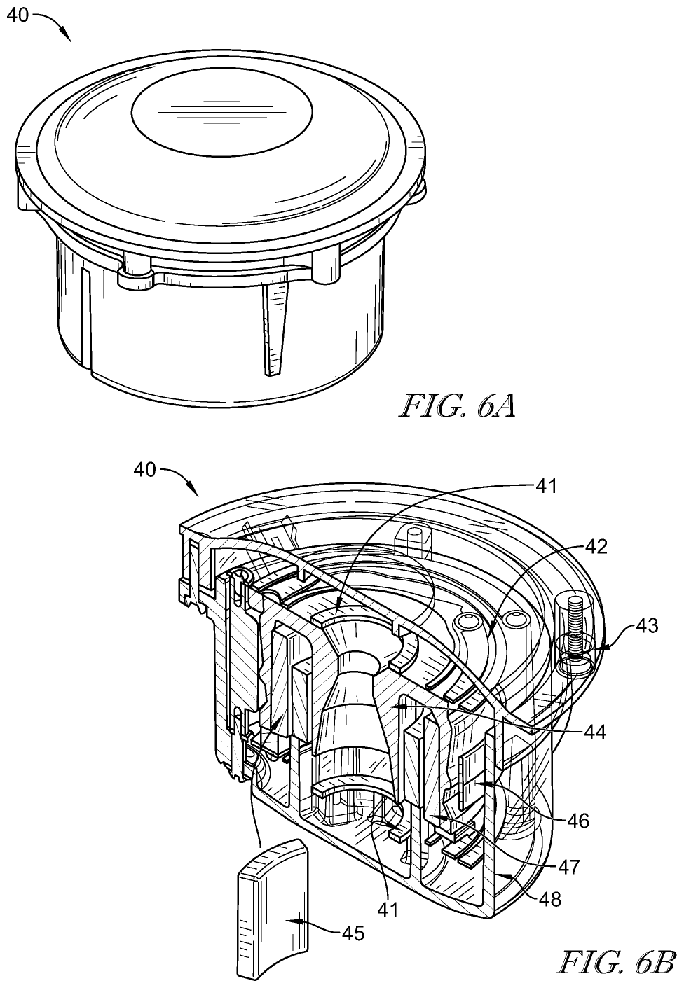

In the illustrative embodiment of FIGS. 1A and 1B, a percussion therapy apparatus 10 positioned on a patient 20 is shown. The percussion therapy apparatus 10 illustratively includes a high frequency chest wall oscillation (HFCWO) therapy vest garment 12 having a chest panel 11 on a first side, a back panel 14 on a second side, and percussive devices 30 that are attachable to the garment 12 to engage with the patient's torso to provide thoracic percussion therapy. The percussive devices 30 may be embodied as a pulsating oscillating disc (POD), also known as a voice coil actuator (VCA). The percussion therapy apparatus 10 illustratively includes an attachment assembly 16 comprising shoulder straps 18 and side strap assemblies 19 for securing the garment 12 to the patient's torso. A voice coil actuator 40 is shown in more detail in FIGS. 6A and 6B. Specifically, the voice coil actuator includes a plurality of axial end-stops 41, a membrane spring 42, a top cover housing 43, a moving backiron 44, a plurality of magnets 45, a mid-ring 46, a coil 47, and a bottom cover housing 48. Further disclosure of a suitable structure for a percussion therapy apparatus 10 is disclosed in U.S. Patent Application Publication No. US2018/0049939 which is incorporated by reference herein in its entirety for the disclosure of structures used to provide high frequency chest wall oscillation (HFCWO) therapy.

A challenge with typical HFCWO therapy is that the devices used must accommodate patients of varying sizes. It has been discovered that in many patients, including those who are treated for bronchiectasis, the impulse force delivered by a particular percussive device 30 may be reduced, thereby reducing costs and the weight of the vest 10. In particular, it has been determined that a reduction in the size of the voice coil actuator 40 used may result in a sufficiently effective HFCWO therapy for patients with bronchiectasis with a corresponding 40% reduction in weight. This is achieved with a therapy that delivers an impulse pulse force of between 3-18 Newton (N) in steps of 3 N. This is significantly reduced from the typical HFCWO therapy system that delivers 25 N of impulse force. The HFCWO therapy for treatment of bronchiectasis delivers impulse forces at a frequency of 5-20 Hz in increments of 1 Hz, allowing the therapy to be tailored to the needs of the specific patient.

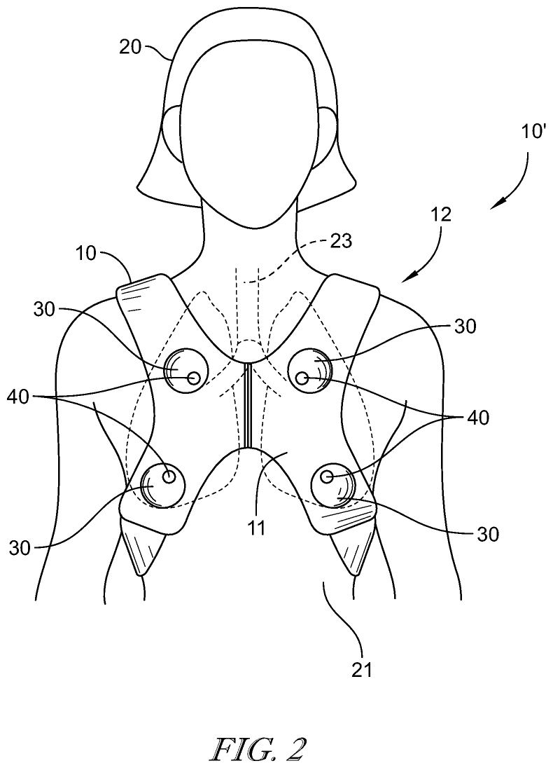

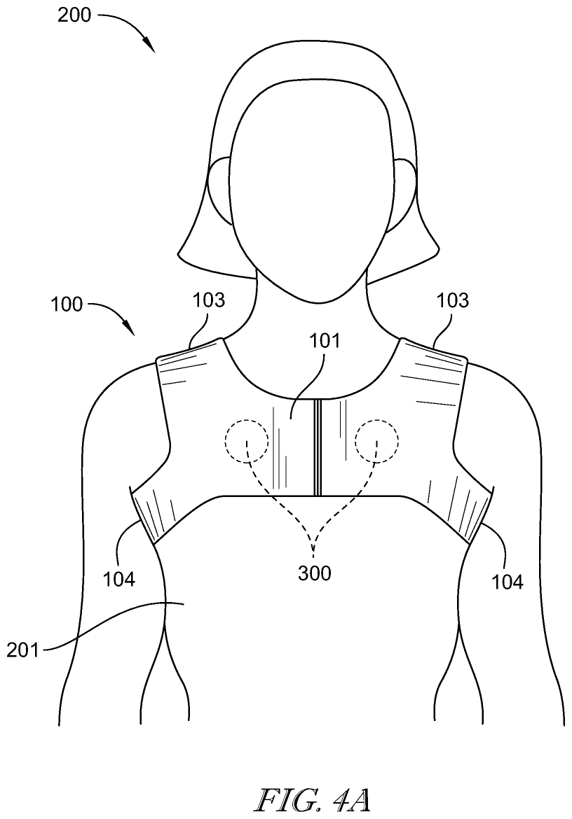

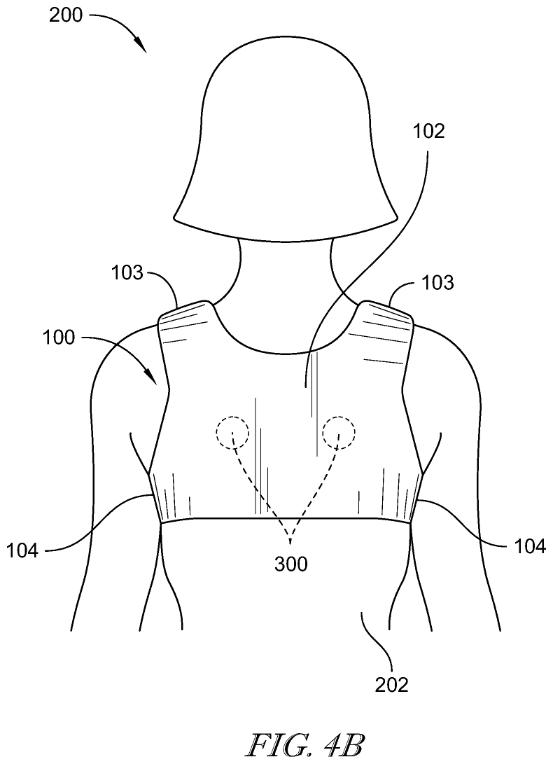

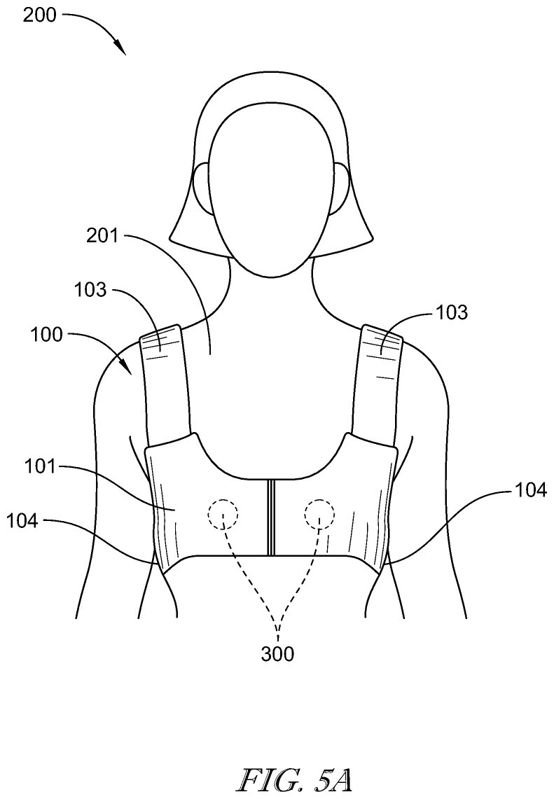

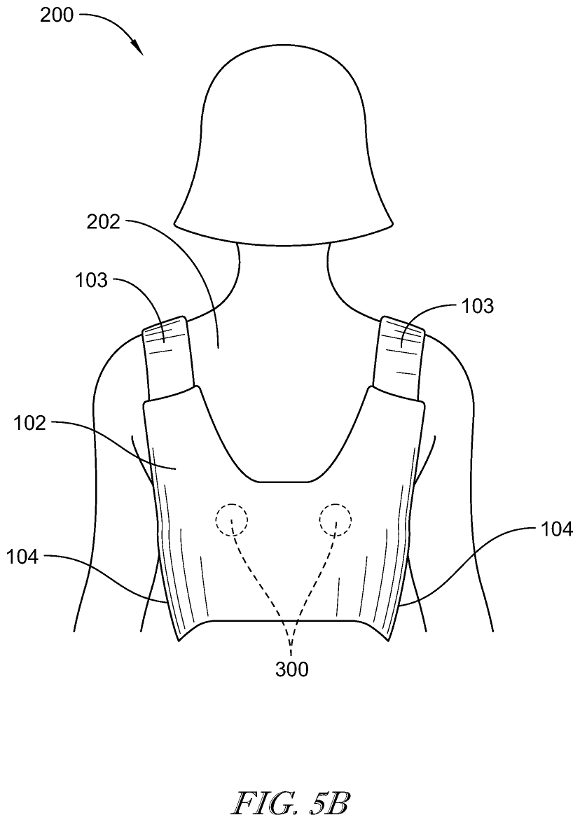

In some embodiments, the garment 12 may further comprise an adjustable strap, described below in FIGS. 4A-4B and 5A-5B. The garment 12 also includes a first side 11 and a second side 12. In the first embodiment of FIGS. 1A and 1B, the first side 11 includes four percussive devices 30 and is positioned over the patient's chest 21, and the second side 12 includes four percussive devices 30 and is positioned over the patient's back 22. The percussive devices 30 on the first side 11 and the second side 12 are oriented over target vibration zones of the patient's respiratory system, described in more detail with reference to FIG. 2. Each percussive device 30 on the first side 11 simulates manual tapping on the patient's chest 21 and each percussive device 30 on the second side 12 simulates manual tapping on the patient's back 22. In some embodiments, the garment 12 may have more than or less than eight percussive devices 30 coupled to it. Furthermore, although there are four percussive devices 30 coupled to either side of the garment 12, it will be appreciated that the garment 12 may have more than four or less than four percussive devices 30 on either side of the garment 12.

The positioning of the garment 12 is illustratively shown in FIG. 2. Specifically, FIG. 2 depicts the first side 11 of the garment 12 and how it is oriented over the patient's respiratory system 23. Two percussive devices 30 on the first side 11 are positioned over the upper part of the patient's respiratory system 23, and two other percussive devices 30 on the first side 11 are positioned over the lower part of the patient's respiratory system 23. Alternative embodiments may include alternate positioning of the pulmonary oscillating discs 30 over the patient's chest 21 and back 22.

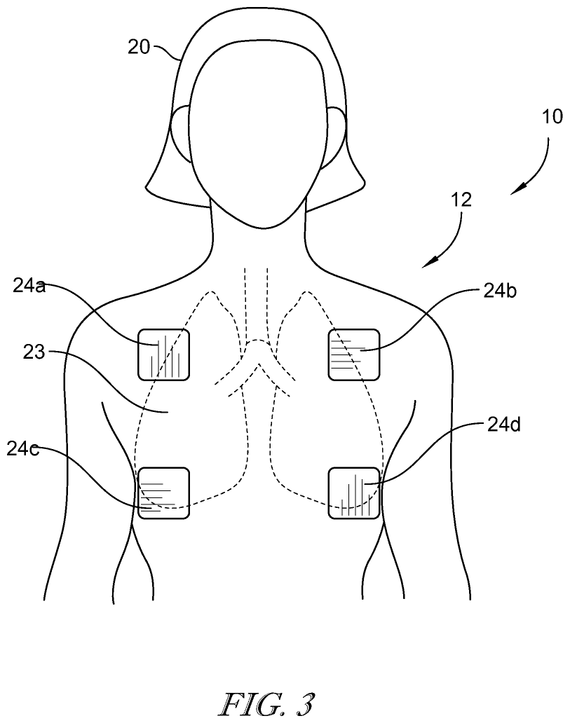

In understanding HFCWO therapy, it is important to understand the interaction between the delivery of HFCWO by various percussive devices 30 and the overall impact on the effectiveness of the therapy. In one empirical study, it has been determined that the influence of the percussive devices 30 varies by their position. An overview of target vibration zones 24 is depicted in FIG. 3. The depicted target vibration zones 24 include a target vibration zone 24a in the upper left of the patient's respiratory system 23, a target vibration zone 24b in the upper right of the patient's respiratory system 23, a target vibration zone 24c in the lower left of the patient's respiratory system 23, and a target vibration zone 24d in the lower right of the patient's respiratory system 23. It was discovered that the delivery of HFCWO therapy only to the target vibrations zones 24a and 24b has a much more significant impact on the mean pulse respiratory flow than that applied to vibration zones 24c and 24d. Thus, in some cases, only applying HFCWO to the target vibration zones 24a and 24b may be sufficient for particular patients.

It is also important to understand that the energy (e) transferred from the chest wall to the lung bronchi at a time (t may be evaluated by integrating the power delivered to the bronchi as follows:

e(t)=∫₀^{t}P(τ)τdτ=Pₘt where Pₘ=mean power

Therefore, the energy required to provide a specific level of therapy can be achieved by either a higher power for a shorter time, or a lower power for a longer time. For example, the energy transferred E by a therapy power Pₘ for a time T can be equivalently achieved by two therapies of half power Pₘ/2 for time 2T In addition, a lower power at a longer time could be more comfortable for patients than with a higher power for a shorter time. Based on these findings, it has been determined that a new approach to HFCWO therapy may be appropriate for some patients.

To that end, FIGS. 4A and 4B, and 5A and 5B show a second embodiment of a HFCWO system 200. In this embodiment, a vest 100 includes adjustable shoulder straps 103 and adjustable underarm straps 104. The first side 101 of vest 100 has two pulmonary oscillating discs 300 and the second side 102 of the vest 100 has two pulmonary oscillating discs 300. In, FIGS. 4A and 4B the vest 100 is in a first position. Like the embodiment shown in FIGS. 1A, 1B, and 2, each pulmonary oscillating disc 300 of this second embodiment includes a magnetic suspended mass and a pair of voice coil actuators (not shown). It should be appreciated that the pulmonary oscillating discs 300 are identical to pulmonary oscillating discs 30. In the depicted first position, the pulmonary oscillating discs 300 on the first side 101 are oriented over the upper part of the patient's chest 201 and the pulmonary oscillating discs 300 of the second side 102 are oriented over the upper part of the patient's back 202. In FIGS. 5A and 5B, the vest 100 is in a second positon. To adjust from the first position to the second position, the shoulder straps 103 and/or the underarm straps 104 are loosened, the vest 100 is repositioned over the patient's chest 201 and back 202, and the shoulder straps 103 and/or the underarm straps 104 are tightened. In the second position, the pulmonary oscillating discs 300 on the first side 101 are oriented over the lower part of the patient's chest 201 and the pulmonary oscillating discs on the second side 102 are oriented over the lower part of the patient's chest 202. Alternatively or additionally, the first side 101 and the second side 102 of the vest 100 could have more than or less than two pulmonary oscillating discs 300 on each side.

In this way, the HFCWO therapy delivered by the system 200 may be tailored for a specific patient by delivering therapy targeted to target vibration zone 24a and 24b only, or by delivering therapy in the first position for a first period of time and in the second position for a second period of time. Based on the variations of the impact of the location of therapy on the patient's mean pulse respiratory flow, the first period of time and the second period of time may be varied to achieve a particular therapeutic benefit.

As shown in FIG. 2, the apparatus 10 has been modified to function as a pleural effusion pre-screening system 10′. FIG. 2 also depicts how a separate sensor 40 is positioned with each percussive device 30. The sensor 40 may be embodied as a sound transducer, digital stethoscope, or microphone. As will be explained in further detail below, the pleural effusion pre-screening system 10′ sequentially and alternately simulates tapping on a patient's chest and determines whether the sounds from the patient's lungs are indicative of the presence pleural effusion. If so, then a caregiver may refer the patient for further diagnostic testing. By integrating this capability into an existing HFCWO system, the ability to regularly monitor for the development of pleural effusion is improved. The zones 24a, 24b, 24c, and 24d to be screened are determined based on areas of the respiratory system 23 that, when tapped, indicate an unusual amount of fluid around the lung. However, it should be appreciated the target vibration zones 24a, 24b, 24c, and 24d can be repositioned based on the anatomy of the patient 20 or other factors such as areas of past treatment. In preferred embodiments, at least one pulmonary oscillating disc 30 is oriented over at least one target vibration zone 24a, 24b, 24c, or 24d of the patient's respiratory system 23.

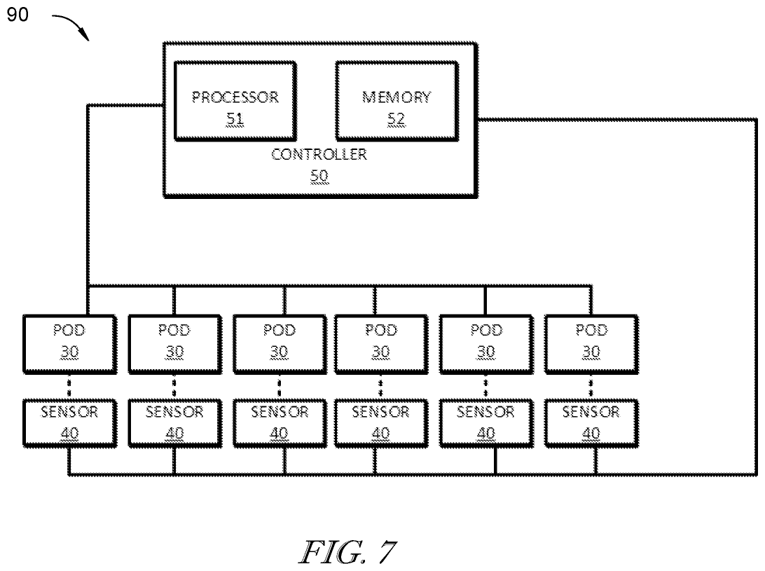

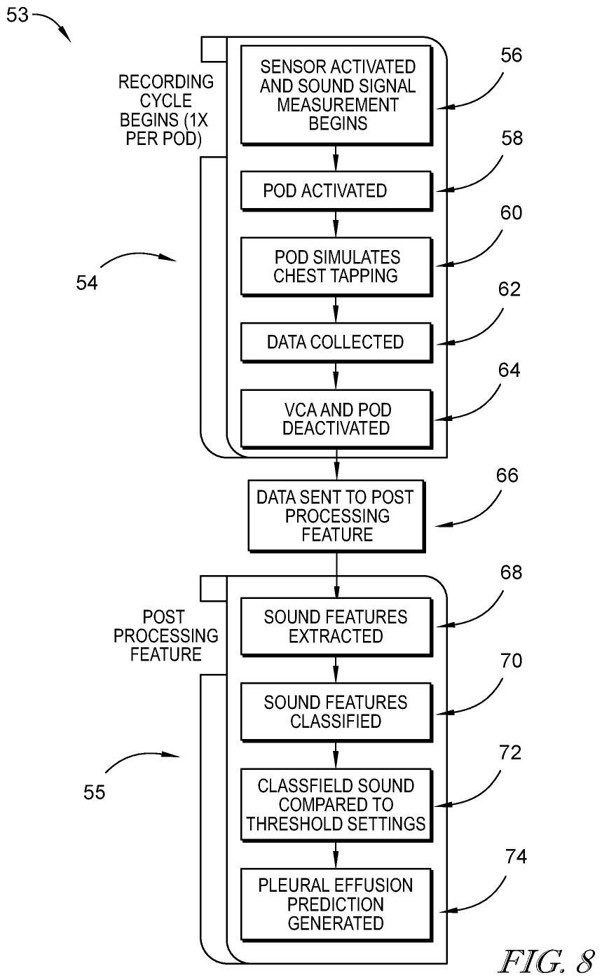

An overview of a control system 90 of the pre-screening pleural effusion system is depicted in FIG. 7. More specifically, garment 12 is positioned on patient 20. Then, a controller 50 is powered on. It should be appreciated that the controller 50 can be coupled or not coupled to the HFCWO vest. The controller 50 comprises a processor 51 and a memory device 52 which includes instructions 53 that, when executed by the processor 51, activates a recording cycle 54 and a post-processing feature 55, described with regard to FIG. 8.

FIG. 8 depicts the instructions 53 in memory device 52 that cause the HFCWO systems of the present disclosure to function as a pleural effusion pre-screening system. When executed, the instructions 53 start with recording process 54. In some embodiments, recording cycle 54 is executed for each percussive device 30 in the garment 12. Alternatively or additionally, recording cycle 54 can be repeated at least twice on one or more percussive devices 30. Furthermore, one or more percussive devices 30 on the garment 12 may not have a recording cycle 54 executed.

Upon initialization, the recording cycle 54 includes activating sensor 40 to begin sound signal measurements at step 56. At step 58, a percussive device 30 is activated and at step 60 the respective percussive device 30 is operated in a manner that simulates manual chest tapping. At step 62, the sound signal measurement data from the sensor 40 is collected. The sensor 40 and percussive device 30 are then deactivated at step 64. As noted on the flow chart of FIG. 8, the process is repeated for each percussive device 30 to collect a full set of data.

At step 66, the data collected from the recording cycle 54 is collected and provided to a post processing feature 55. In some embodiments, the sound signals may be pre-processed applying various digital signal processing techniques known in the art. Sound features 68 are extracted at step 68. Those sound features are then classified at step 70. The classification can be accomplished through machine learning or through traditional discriminate analysis. The classified sound features are compared to threshold settings at step 72.

Based on the comparison to the threshold values, a likelihood of the presence of pleural effusion is established at step 74. The likelihood of the presence of pleural effusion is then communicated to the caregiver by a user interface 80. Various approaches to lung sound processing, classification, and threshold comparisons are provided in: Oletic, Dinko, et al. “Low-Power Wearable Respiratory Sound Sensing.” Sensors, vol. 14, no. 4, 2014, pp. 6535-6566., https://doi.org/10.3390/s140406535; Khan, Sibghatullah I. “Respiratory Sound Analysis for Identifying Lung Diseases: A Review”, International Journal of Science and Research (IJSR), Volume 3 Issue 11, November 2014, pp. 566-571.; Palaniappan, Rajkumar, et al. “Machine Learning in Lung Sound Analysis: A Systematic Review.” Biocybernetics and Biomedical Engineering, vol. 33, no. 3, 2013, pp. 129-135., https://doi.org/10.1016/j.bbe.2013.07.001; H. Wang, et al. “Lung sound/noise separation in anesthesia respiratory monitoring.” WSEAS Transactions on Systems, Vol. 3, June 2004, pp. 1839-1844.; and Gurung, Arati, et al. “Computerized Lung Sound Analysis as Diagnostic Aid for the Detection of Abnormal Lung Sounds: A Systematic Review and Meta-Analysis.” Respiratory Medicine, vol. 105, no. 9, 2011, pp. 1396-1403., https://doi.org/10.1016/j.rmed.2011.05.007.

Although this disclosure refers to specific embodiments, it will be understood by those skilled in the art that various changes in form and detail may be made without departing from the subject matter set forth in the accompanying claims.

1. A pleural effusion pre-screening system comprising:
a garment wearable by a patient,
a first percussion device secured to the garment and positioned in a predetermined location relative to the body of the patient,
a first sensor secured to the garment and positioned in the predetermined location relative to the body of the patient, and
a controller including a processor and a memory device including instructions that, when executed by the processor, activates the first sensor to begin recording sound data from the patient's respiratory system, activates the first percussion device to simulate manual chest tapping, collects sound data from the patient's respiratory system, and sends the sound data to a post processing feature.

2. The pleural effusion pre-screening system of claim 1, wherein the sensor is a sound transducer.

3. The pleural effusion pre-screening system of claim 1, wherein the sensor is a microphone.

4. The pleural effusion pre-screening system of claim 1, wherein the sensor is a digital stethoscope.

5. The pleural effusion pre-screening system of claim 1, further comprising a plurality of percussion devices and a plurality of sensors associated with each of the percussion devices.

6. The pleural effusion pre-screening system of claim 5, wherein the memory device includes further instructions that, when executed by the processor, activates a second sensor to begin recording sound data from the patient's respiratory system, activates a second percussion device to simulate manual chest tapping, collect sound data from the patient's respiratory system, deactivates the second sensor and the second percussion device, and sends the sound data from the second sensor to a post processing feature.

7. The pleural effusion pre-screening system of claim 5, wherein the memory device includes further instructions that, when executed by the processor, sequentially activates each additional sensor to begin recording sound data from the patient's respiratory system, activates an additional percussion device associated with the respective additional sensor to simulate manual chest tapping, collects sound data from the patient's respiratory system, deactivates the additional sensor and the additional percussion device, and sends the sound data from the additional sensor to a post processing feature.

8. The pleural effusion pre-screening system of claim 7, wherein the memory device includes further instructions that, when executed by the processor, processes the sound data to extract sound features, classifies the sound features, compares the classified sound features to threshold settings, and, based on the comparison of the sound features to the threshold settings, predicts the likelihood that the patient is experiencing pleural effusion.

9. The pleural effusion pre-screening system of claim 6, wherein the memory device includes further instructions that, when executed by the processor, processes the sound data to extract sound features, classifies the sound features, compares the classified sound features to threshold settings, and, based on the comparison of the sound features to the threshold settings, predicts the likelihood that the patient is experiencing pleural effusion.

10. The pleural effusion pre-screening system of claim 6, wherein the memory device includes further instructions that, when executed by the processor, applies a digital signal filter to the sound data prior to performing the extraction of sound features.

11. A high frequency chest wall oscillation (HFCWO) system comprising
an adjustable garment configured to be positioned on a patient,
at least one first percussion device positioned on a first side of the garment, and
at least one second percussion device positioned on a second side of the garment,
wherein the garment is adjustable between a first position locating the first and second percussion devices to target an upper zone of the patient's lungs and a second position locating the first and second percussion devices to target a lower zone of the patient's lungs.

12. The HFCWO system of claim 11, wherein the first and second percussion devices are operable to vary the power delivered to the patient's lungs such that a first power can be delivered at each respective zone.

13. The HFCWO system of claim 12, wherein the adjustable garment includes an adjustable strap.

14. The HFCWO system of claim 13, wherein the adjustable strap comprises at least one adjustable shoulder strap and at least one adjustable underarm strap.

15. The HFCWO system of claim 14, further comprising
a sensor associated with each respective percussion device, and
a controller including a processor and a memory device including instructions that, when executed by the processor, sequentially and independently activates each sensor to begin recording sound data from the patient's respiratory system, activates the percussion device associated with the respective sensor to simulate manual chest tapping, collects sound data from the patient's respiratory system, deactivates the additional sensor and the additional percussion device, repeats the sequential and independent activation of the sensor, respective percussion device, data collection and deactivation of the sensor and respective percussion device for each set of sensors and percussion devices, and sends the sound data from the additional sensor to a post processing feature.

16. The HFCWO system of claim 15, wherein the memory device includes further instructions that, when executed by the processor, processes the sound data to extract sound features, classifies the sound features, compares the classified sound features to threshold settings, and, based on the comparison of the sound features to the threshold settings, predicts the likelihood that the patient is experiencing pleural effusion.

17. The HFCWO system of claim 16, wherein the memory device includes further instructions that, when executed by the processor, applies a digital signal filter to the sound data prior to performing the extraction of sound features.

18. The HFCWO system of claim 15, wherein the memory device includes further instructions that, when executed by the processor, applies a digital signal filter to the sound data prior to performing the extraction of sound features.

19. The HFCWO system of claim 11, wherein the percussion devices administer a therapy force of no more than 18 Newton at a frequency no more than 20 Hertz.

20. The HFCWO system of claim 11, wherein the therapy force is administered at a range of 3 to 18 Newton and the frequency of the therapy force is at a range of 5 to 20 Hertz.
